(19) United States
(12) Patent Application Publication
(10) Pub. No.: US 2014/0207017 A1
(43) Pub. Date: Jul. 24, 2014

(54) SIGNAL QUALITY MONITOR FOR ELECTROMYOGRAPHIC SENSORS

(71) Applicant: Altec, Inc., (US)

(72) Inventors: L. Donald GILMORE, Wellesley, MA (US); Carlo J. DELUCA, Wellesley, MA (US)

(73) Assignee: ALTEC, INC., Boston, MA (US)

(21) Appl. No.: 13/747,882

(22) Filed: Jan. 23, 2013

(51) Int. Cl. A61B 5/00; A61B 5/04; A61B 5/0488

(52) U.S. Cl. CPC A61B 5/7207; A61B 5/0488; A61B 5/04012; A61B 5/743
USPC 600/546

(57) ABSTRACT

A process for assessing the signal quality of electromyographic (EMG) sensor signal data outputs based of a set of time domain and frequency domain signal performance metrics, including the magnitude of the baseline noise, line interference power spectra, and signal to baseline noise ratio parameters of the signal, whose calculated values are compared with a set of pre-defined acceptable values to determine whether or not the signal is of acceptable signal quality, and provide a pass/fail output. A graphic and text display output is provided to visually indicate the result. The visual display includes the presentation of the calculated parameter values of the baseline noise magnitude, line interference magnitude, and signal to baseline noise magnitude, using a combination of digital and analog indicators, each marked with the respective value of their pre-defined performance metric. The visual display includes the presentation of descriptive text blocks associated with each displayed parameter, with relevant instructions for addressing and correcting conditions of unacceptable signal quality.

### TECHNICAL FIELD

This application relates to the field of sensing bio-potentials generated within a living body and more particularly, relates to assessment of signal quality of sensors used for detecting electrical activity from muscles using electromyographic (EMG) signals.

### BACKGROUND

Depolarization potentials created during a muscle fiber contraction generate an electrical field gradient that propagates in a direction along the fibers throughout the volume conductor that includes the muscle, the surrounding tissue, and skin layers. Indwelling needle or fine wire electrodes placed within the muscle tissue or electrodes placed on the surface of the skin allow for the detection of this electrical field gradient providing the temporal summation of the propagating depolarization potentials of the active muscle fibers in the underlying vicinity of the electrode. Signal potentials emanating from the muscle tissue are conveyed via ionic transport through the tissue's electrolytes to the exposed conductive contact surfaces of the electrode. The resulting voltage is the electromyographic (EMG) signal.

Applications for using EMG signal measurement are diverse and can range from sports and ergonomic activities to the clinical evaluation of patients and neuromuscular research applications which investigate motor control. Analysis of the EMG signal can provide valuable information about muscle performance not obtainable by other means. The EMG signal amplitude may be representative of the force generated by the muscle, which, unlike externally applied force measuring devices, can be used to assess the individual force contributions from a group of muscles acting together on a joint. Multiple EMG sensors placed on the limbs can monitor muscle activity levels and coordination during, for example, gait studies or studies of neurological disorders such as Parkinson's disease. Changes in the frequency spectra of the EMG signal resulting from localized muscle fatigue can be used to more objectively assess appropriate activity levels and durations of tasks in the workplace. Specialized sensors developed to measure the firing patterns of the individual motor units of the EMG signal can be used to investigate motor control. Analysis of EMG signal temporal and spectral parameters may therefore be useful in all these investigations.

However, the voltage amplitude of EMG component of the signal detected by a sensor is inherently small, typically ranging from ten microvolts to several millivolts. In addition to the EMG signal component, the voltage at the sensor output includes the contribution from other noise sources generated by the inherent noise of the sensor's electronics, the electrolytic interface established between the metallic contacts of the sensor and intervening tissue, voltages induced by external power line sources, and voltages induced from the movement of the sensor contacts with respect to the intervening tissue. The summation of these intrinsic and extrinsic noise source components may be termed the baseline noise voltage.

The state of the art EMG sensor design minimizes the contribution of electronically generated circuit noise, however the predominate baseline noise sources relate to the sensor/skin tissue interface and are a function of contact design, skin preparation, and location of interference sources in the vicinity of the sensor. Variability in an individual's skin type and the amount of moisture and oils on the skins surface at different locations on the body affect the quality of the sensor/skin tissue interface and the resultant amount of baseline noise generated. This effect can be especially problematic when the electrolytic skin interface exhibits high impedance resulting from the lack of suitable moisture between the electrode contacts and the skin. This impedance can reach tens of meg-ohms for contacts with an area of 1 mm squared placed on unprepared skin. Additionally, power lines and electrical equipment operating in the vicinity of the sensor may induce an undesirable level of 50/60 Hz frequency components and related harmonics into the sensor output if the sensor has been improperly applied to the skin. The contribution from all these baseline noise source components can be a large percentage of the overall signal output, especially for low level muscle contractions with low EMG signal amplitude, or sensor locations above muscles with large amounts of intervening adipose tissue.

The relationship between the level of EMG signal and the amount of baseline source noise is expressed as the signal to baseline noise ratio (SNR) where a higher SNR value indicates higher EMG signal quality. EMG signals with low SNR values can prove problematic in many applications that determine muscle performance using temporal and spectral analysis of the EMG signal. Minimizing the sensor/skin tissue interface noise and power line induced noise sources of the EMG signal baseline, while maximizing the available EMG signal amplitude by properly locating the sensor on the muscle ensures an acceptable SNR.

In many research based applications utilizing EMG, signal quality is assessed by visual observation of the EMG signal trace during data acquisition. Visual observation of EMG signal and the interpretation of signal quality by its nature are subjective, and dependent on the experience of a trained observer. The observer first estimates the amplitude of the baseline envelope and any periodic interference components of the sensor output during the quiescent state. The amplitude of the EMG signal during a contraction is then noted, and the signal quality is determined based on these observations. This procedure is repeated for each attached sensor. Determining signal quality in this way may be acceptable for preliminary investigations where there is both adequate time and personnel. However, for clinical research applications, this approach may prove cumbersome for protocols where multiple tests are performed with multiple sensors. Furthermore, there are situations where it may be difficult to visually distinguish the level of periodic interference components within the baseline noise envelope and the SNR of low level EMG signals. Most signal quality tests are performed when the sensor is initially applied at the outset of the data acquisition and may not be repeated during subsequent trials, increasing the risk of poor data quality.

Some clinically based EMG systems such as those specifically designed for applications to measure EKG with separate, individually applied skin electrode contacts have features which automatically determine baseline signal quality by measuring the impedance of each contact. A low impedance reading indicates that the contact is attached to the skin. High impedance indicates that there is poor contact or that the electrode has become detached and a warning condition is issued. These clinical devices measure signal quality as it relates specifically to monitoring the EKG signal, however, the EKG signal is not representative of the temporal and spectral characteristics of EMG signals obtained with differential sensors during voluntary muscle contractions. Voluntary contractions associated with EMG signals can vary in amplitude as function of contraction level from baseline noise to maximum voluntary contraction depending on the selected contraction profile. The EMG signal amplitude is at least an order of magnitude lower than the EKG signal. The level of sensor/skin tissue interface and power line induced baseline noise sources, which may not be a significant issue for monitoring EKG signals, could be problematic for monitoring low level voluntary EMG signals. Because of the greater frequency bandwidth (up to 500 Hz) of voluntarily elicited EMG signals, the effect of baseline noise with higher frequency components may be a more predominate factor in determining signal quality than in EKG applications. And while using impedance measurement to determine signal quality might seem a useful technique for voluntary EMG measurements, it can prove problematic, especially for differential pair sensors with a 1 cm to 2 cm inter-electrode spacing. As a rule, the lower the electrode contact impedance with the skin, the lower the baseline noise value. With a differential inter-electrode spacing used by sensors designed for voluntary EMG measurements, a low electrode contact impedance reading could be interpreted as a properly applied sensor with low baseline noise. However, a low impedance reading could also mean that there is excessive moisture acting as an electrical short between the contacts which would attenuate the EMG signal, leading to a lower than expected SNR. Therefore for sensors used in voluntary EMG measurements, the determination of the actual baseline noise value, combined with assessment of SNR is superior to impedance measurement in determining inter-electrode shorting due to excessive moisture.

The signal quality assessment techniques utilized by clinical devices such as EKG monitors may have limited applicability for use in measurement of signal quality for voluntarily elicited EMG signals because EKG assessment techniques (and similar) do not provide a quantifiable indication of the output levels of baseline noise, power line interference, and SNR to the user. It is the unique combination of these parameters which provides a comprehensive assessment of signal quality in applications utilizing voluntarily elicited EMG signals. Some existing designs of EMG equipment address the issue of signal quality by utilizing electronic circuit hardware to measure the EMG signal saturation (clipping) and the level of power line interference contamination. The equipment may provide visual and audio output by lights and buzzers when a hardwired limit is exceeded, but does not measure the baseline noise value or compute the SNR value, and may lack the ability to perform spectral analysis to determine the level of harmonically related periodic components of the power line frequency. While a useful aid in finding catastrophic failure conditions such as sensor detachment, such equipment may lack the sophistication of providing a complete interpretation of signal quality.

All of the aforementioned configurations offer only a limited set of solutions for determining EMG signal quality required as a prerequisite for the successful execution of a given clinical or research EMG application utilizing the measurement and analysis of voluntary EMG signal. It would be useful to provide an automatic EMG sensor signal quality assessment process that would ensure the acquisition of data with proper signal quality necessary for the successful execution of a clinical or research EMG application.

### SUMMARY OF THE INVENTION

According to the system described herein, evaluating at least one of a plurality of EMG sensor signal data outputs includes determining regions of baseline noise, line interference, and summated motor unit action potential components for each of the plurality of the signal data outputs, arithmetically combining corresponding time and frequency domain parameters of each region into parameters to provide a set of calculated electromyographic signal performance metrics that include a baseline noise value, a magnitude and power spectra of a summated motor unit action potential components, a line interference spectra value, an EMG signal to baseline noise ratio, and a maximum data value, visually displaying the calculated electromyographic signal performance metrics, comparing the calculated electromyographic signal performance metrics with a set of pre-defined electromyographic signal performance metrics values, providing an output indicating an acceptable signal quality result in response to all of the calculated performance metrics meeting the pre-defined electromyographic signal performance metrics, and providing an output indicating a non-acceptable signal quality result in response to at least one of the calculated performance metrics not meeting the pre-defined performance metrics. There may be one EMG sensor signal data output or there may be more than one EMG sensor signal data output. The set of pre-defined electromyographic signal performance metrics may be determined according to performance requirements of a selected EMG application, and may include an allowable number of segments from a baseline region containing motor unit action potentials, an allowable value for the EMG signal to baseline noise ratio, an allowable maximum value for a signal data output, values of coefficients of variables used in mathematical functions that arithmetically combine a plurality of individual parameter values into single respective parameter values, values of coefficients of variables used in a mathematical function that calculates an allowable baseline noise value based on the EMG signal to baseline noise ratio, and values of coefficients of variables used in a mathematical function that calculates an allowable value for spectral components of the line interference based on a calculated value of the EMG signal to baseline noise ratio and a calculated value of the summated motor unit action potential components power spectra. Determination of the summated motor unit action potential component may include an algorithm designed to identify data segments containing motor unit action potentials and calculate a summated motor unit action potential components region signal envelope and where the magnitude of the summated motor unit action potential components region signal envelope may include in the calculated performance metrics. Determination of the line interference component may include an algorithm designed to calculate a power spectral density function of the baseline noise and to identify magnitudes of 50 Hz, 60 Hz, and associated harmonic components of the EMG sensor signal data and where the magnitudes of the components of line interference may be included in the calculated performance metrics. Determining regions of baseline noise may include an algorithm designed to divide a signal envelope of the summated motor unit action potential component by a baseline noise region signal envelope and provide a result thereof as the signal to baseline noise ratio where a magnitude of the signal to baseline noise ratio may be included in the calculated performance metrics. Determination of the maximum data value may include an algorithm designed to calculate a maximum absolute value of the data and the maximum absolute value may be included in the calculated performance metrics. The calculated performance metrics may include a mean value of a magnitude of a baseline noise region signal envelope, a mean value of a line interference spectra magnitude, and a mean value of the signal to baseline noise ratio, where each of the mean values may be calculated from a plurality of individual parameter values. An allowable value for a baseline noise signal envelope may be calculated as a function of a pre-defined value of allowable baseline noise and the calculated signal to noise ratio. An allowable value for the spectral components of the line interference may be calculated as a function of a pre-defined value of allowable spectral components of the line interference and the calculated signal to noise ratio. A compliance state of a recorded signal quality result output may be provided as an accessible digital control output available for integration with other hardware and software processes. A visual graphic display presentation of results output may be expanded by user activated control to include presentation of calculated parameter values of the baseline noise, line interference, and signal to baseline noise ratio, using a combination of digital and analog indicators, each marked with a value of the pre-defined performance metric for respective output parameters thereof, where the visual graphic display presentation may include presentation of additional descriptive text blocks associated with each respective calculated parameter value output. The visual graphic display presentation of results output may be automatically expanded to include presentation of calculated parameter values of the baseline noise magnitude, line interference magnitude and signal to baseline noise magnitude using a combination of digital and analog indicators, each marked with the value of the pre-defined performance metric for respective output parameters thereof. The visual graphic display may include presentation of additional descriptive text blocks associated with each respective calculated parameter value output having text content based on context determined by a state of compliance with a set of pre-defined performance metrics values. The descriptive text blocks may provide instructions for addressing and correcting conditions of non-compliance of each calculated parameter value output with respect to the set of pre-defined performance metrics values.

According further to the system described herein, computer software, provided in a non-transitory computer-readable medium, evaluates at least one of a plurality of EMG sensor signal data outputs. The software includes executable code that determines regions of baseline noise, line interference, and summated motor unit action potential components for each of the plurality of the signal data outputs, executable code that arithmetically combines corresponding time and frequency domain parameters of each region into parameters to provide a set of calculated electromyographic signal performance metrics that include a baseline noise value, a magnitude and power spectra of a summated motor unit action potential components, a line interference spectra value, an EMG signal to baseline noise ratio, and a maximum data value, executable code that visually displays the calculated electromyographic signal performance metrics, executable code that compares the calculated electromyographic signal performance metrics with a set of pre-defined electromyographic signal performance metrics values, executable code that provides an output indicating an acceptable signal quality result in response to all of the calculated performance metrics meeting the pre-defined electromyographic signal performance metrics, and executable code that provides an output indicating a non-acceptable signal quality result in response to at least one of the calculated performance metrics not meeting the pre-defined performance metrics. The set of pre-defined electromyographic signal performance metrics may be determined according to performance requirements of a selected EMG application, and may include an allowable number of segments from a baseline region containing motor unit action potentials, an allowable value for the EMG signal to baseline noise ratio, an allowable maximum value for a signal data output, values of coefficients of variables used in mathematical functions that arithmetically combine a plurality of individual parameter values into single respective parameter values, values of coefficients of variables used in a mathematical function that calculates an allowable baseline noise value based on the EMG signal to baseline noise ratio, and values of coefficients of variables used in a mathematical function that calculates an allowable value for spectral components of the line interference based on a calculated value of the EMG signal to baseline noise ratio and a calculated value of the summated motor unit action potential components power spectra. A compliance state of a recorded signal quality result output may be provided as an accessible digital control output available for integration with other hardware and software processes. The system may also include executable code that provides a visual graphic display presentation of results output that are expanded by user activated control to include presentation of calculated parameter values of the baseline noise, line interference, and signal to baseline noise ratio, using a combination of digital and analog indicators, each marked with a value of the pre-defined performance metric for respective output parameters thereof, where the visual graphic display presentation includes presentation of additional descriptive text blocks associated with each respective calculated parameter value output.

The system described herein is a process for assessing the signal quality of EMG sensor signal data outputs based of a set of signal performance metrics including the magnitudes of the baseline noise, line interference power spectra, and signal to baseline noise ratio parameters of the signal, whose calculated values are compared with a set of pre-defined acceptable values to determine whether or not the signal is of acceptable signal quality and output a pass/fail result. A graphic and text display output is provided to visually indicate the results. The visual display includes the presentation of the calculated parameter values of the baseline noise magnitude, line interference magnitude, and signal to baseline noise magnitude, using a combination of digital and analog indicators, each marked with the respective value of their pre-defined performance metric. The visual display includes the presentation of descriptive text blocks associated with each displayed parameter, with relevant instructions for addressing and correcting conditions of unacceptable signal quality.

The system described herein relates to an improved mechanism for automatically determining whether or not the quality of the signal data acquired from EMG sensors is suitable for successful execution of a clinical or research EMG application and, if necessary, suggests a sequence of corrective actions that should be taken in order to ensure the acquisition of data with proper signal quality. The system described herein is applicable to all EMG sensor technologies including indwelling sensors such as needle and fine wire electrodes, as well as bipolar and multi-contact surface array sensors. The system described herein is unique in that it takes into consideration the compounding effect of the multiple intrinsic and extrinsic factors which influence the quality of the EMG signal.

The system described herein may be applied to one or more EMG sensor signal data outputs. The system described herein may provide real-time output of signal quality during data acquisition. The system described herein may be included as one of the steps in a sequence of steps defining a protocol used to acquire data. The system described herein may be included as one of the steps in a sequence of steps defining a protocol used to analyze the acquired data. The system described herein may determine regions of baseline noise and regions EMG signal activity within the acquired data. The EMG signal activity may be the result of voluntary muscle contraction. The system described herein may combine the respective calculated the time and frequency domain parameters of selected regions of baseline noise and EMG activity simultaneously acquired from each output of a group of multiple EMG sensor signal data outputs, to create a representative value for each parameter. The time domain parameters may include the magnitude of the baseline noise envelope, the magnitude of the EMG activity envelope, the maximum absolute value of the acquired data, and time stamps defining the locations of the selected regions of baseline noise and signal activity within the acquired data. The frequency domain parameters may include the magnitude of selected line interference frequency components of the power density spectrum of the selected regions of baseline noise, and the median or mean frequency of the power density spectrum of the selected regions of EMG signal activity within the acquired data. The system described herein may calculate the time and frequency domain parameters of selected regions of baseline noise and EMG activity within the acquired data. The system described herein may divide the magnitude of the envelope of the selected regions of EMG signal activity by the magnitude of the envelope of the selected regions of baseline noise and expresses the calculated result as the signal to noise ratio. The system described herein may allow the value of the magnitude of the envelope of the selected regions of baseline noise may be a function of the calculated the signal to noise ratio. The allowable value of the magnitude of selected line interference frequency components of the power density spectrum may be a function of both the calculated the signal to noise ratio and the median or mean frequency of the selected regions of EMG signal activity. The calculated time and frequency parameters of the baseline and EMG signal regions, together with the calculated signal to noise ratio, may form a set of calculated signal quality performance metrics. The system described herein may use a multi-parameter determination algorithm to compare a set of the calculated values of the signal quality performance metrics with a set of pre-defined EMG signal performance metrics values to provide an output indicating an acceptable signal quality result when all the calculated performance metrics meet or exceed the pre-defined performance metrics, and may provide an output indicating a non-acceptable signal quality result when any of the calculated performance metrics do not meet the pre-defined performance metrics. The output of the system described herein indicating the state of compliance with the set of pre-defined performance metrics may be a Boolean value that can be used for conditional control. The system described herein may provide a visual display output of the system described herein indicating the state of compliance with the set of pre-defined performance metrics. The system described herein may provide visual display presentation of additional descriptive text blocks associated with each respective calculated parameter value output, whose text content is based on the context determined by the state of compliance with the set of pre-defined performance metrics values. The system described herein may provide visual display presentation of descriptive text blocks provide instructions for addressing and correcting conditions of non-compliance of each calculated parameter value output with respect to the set of pre-defined performance metrics values. The visual display presentation of results output may be optionally expanded by user activated control, to include the presentation of the calculated parameter values of the baseline noise magnitude, line interference magnitude, and signal to baseline noise magnitude, using a combination of digital and analog indicators, each marked with the value of the pre-defined performance metric for their respective output parameter. The visual display presentation of results output may be automatically expanded during the output state of non-compliance with the set of pre-defined performance metrics, to include the presentation of the calculated parameter values of the baseline noise magnitude, line interference magnitude and signal to baseline noise magnitude using a combination of digital and analog indicators, each marked with the value of the pre-defined performance metric for their respective output parameter.

The system described herein replaces the subjective interpretation of signal quality based on visual observation or based on preset hardware based limits, with a more comprehensive series of software based algorithms which parameterize and integrate the multiple extrinsic and intrinsic factors which affect electromyographic signal quality. These parameters are compared with a knowledge base of pre-defined metrics established for a given application to determine a pass/fail result. The visual graphic outputs of data and instructive text display outputs provided by the assessment further simplify the task of correcting conditions indicating poor signal quality. The technique could be applied to all types of sensor technologies used to acquire the EMG signal, including those incorporating needle, fine wire, bar and pin electrodes, as well as multiple sensor arrays.

### BRIEF DESCRIPTION OF THE DRAWINGS

These and other objects and features of the invention will become more apparent upon perusal of the following description taken in conjunction with the accompanying drawings wherein:

FIG. 1 is a system block diagram illustrating an embodiment of the system described herein;

FIG. 2 is a block diagram of a baseline noise and EMG signal activity region selection algorithm according to an embodiment of the system described herein;

FIG. 3A is a plot of a selected baseline region according to an embodiment of the system described herein;

FIG. 3B is a plot of a selected baseline region with motor unit pulses according to an embodiment of the system described herein;

FIG. 4 is a block diagram illustrating baseline noise MU removal according to an embodiment of the system described herein;

FIG. 5A is a time domain plot of a baseline noise and EMG signal activity regions of an EMG sensor signal data output according to an embodiment of the system described herein;

FIG. 5B shows a plot of a calculated Baseline Noise Power Density Spectrum plot according to an embodiment of the system described herein;

FIG. 5C shows an EMG Power Density Spectrum plot according to an embodiment of the system described herein;

FIG. 6A is an expanded view of a selected baseline region of a signal according to an embodiment of the system described herein;

FIG. 6B is a Power Density Spectrum plot of a signal baseline noise signal according to an embodiment of the system described herein;

FIG. 7A is an expanded view of a selected baseline noise region of a signal contaminated with power line interference according to an embodiment of the system described herein;

FIG. 7B is a Power Density Spectrum plot of a baseline signal contaminated with power line interference according to an embodiment of the system described herein;

FIG. 8 is an expanded view of EMG signal activity region where portions of a signal are clipped at maximum data values according to an embodiment of the system described herein;

FIG. 9 is a block diagram illustrating performance metrics comparison matrix selection according to an embodiment of the system described herein;

FIG. 10 is an example screen shot of a video display of a signal quality assessment indicating a Pass result according to an embodiment of the system described herein;

FIG. 11 is an example screen shot of a video display of a signal quality assessment indicating a Fail result according to an embodiment of the system described herein;

FIG. 12 is a block diagram of another embodiment for processing multiple signal data channels according to an embodiment of the system described herein;

FIG. 13 is a block diagram of baseline noise and EMG signal activity region selection algorithm according to an embodiment of the system described herein;

FIG. 14 is a block diagram of an algorithm used to combine multiple calculated data outputs according to an embodiment of the system described herein;

FIG. 15 is an example screen shot of a video display of a signal quality assessment for multiple signal data channels indicating a Pass result according to an embodiment of the system described herein;

FIG. 16 is an example screen shot of a video display of a signal quality assessment for multiple signal data channels indicating a Fail result according to an embodiment of the system described herein;

### DESCRIPTION OF VARIOUS EMBODIMENTS

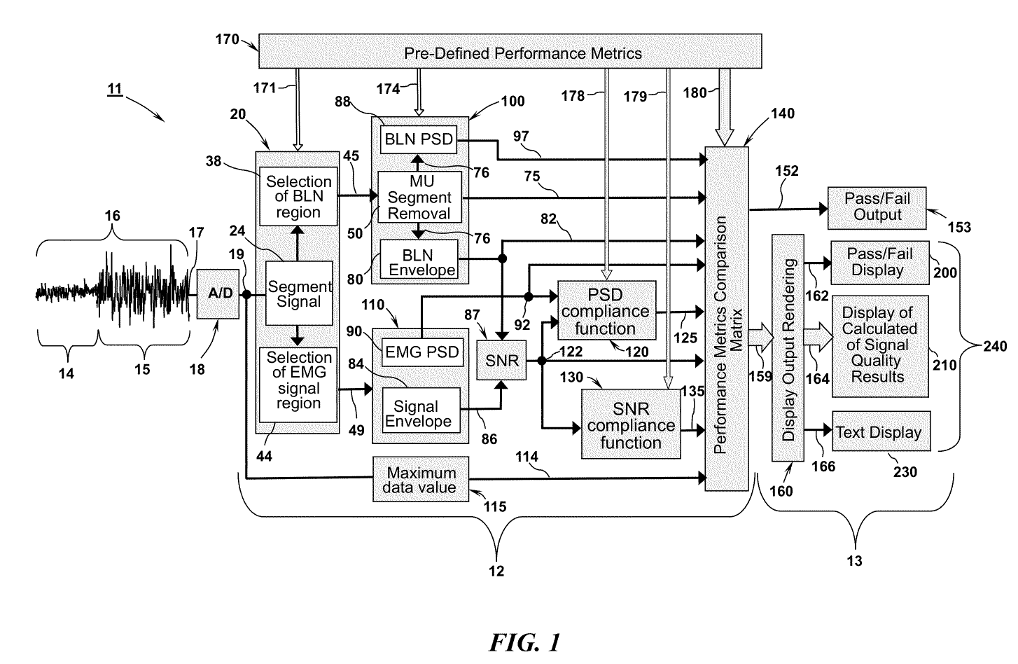

Referring to FIG. 1, a system 11 assesses signal quality of EMG sensor signal data outputs using a set of signal quality compliance algorithms 12, generated visual graphics and text displays 13, and a digital pass/fail result output 153. The sensor data may be from a single differential EMG sensor or from a double differential EMG sensor placed on the skin of a subject. The system 11 may be implemented using hardware (e.g., off-the shelf computer processing hardware), software (e.g., a computer program written in an appropriate language to provide the functionality described herein), or some combination thereof. In an embodiment herein, the system 11 may be implemented using a computing device such as an off-the-shelf personal computer running an appropriate operating system and/or may be or may include an embedded system running in connection with a personal computer or a relatively larger computing device, such as a minicomputer. The computing device receives signal data in the form of real-time analog or digital inputs or, in some cases, may process data that has already been accumulated and stored and provided on analog or digital tape or otherwise provided as analog or digital data. The data may be provided by band pass filtering sensor data, possibly providing pre-amplification with gain and filtering. In the case of analog data, an analog to digital converter may be used. The computing device is programmed to provide the functionality described herein.

The set of signal quality compliance algorithms 12 includes a baseline noise and EMG signal region selection algorithm 20, a group of baseline noise (BLN) region signal processing algorithms 100, a group of EMG signal processing algorithms 110, a maximum data value algorithm 115, an SNR computation algorithm 87, a PSD compliance function 120, an SNR compliance function 130, and a performance comparison matrix 140. In operation, pre-defined signal quality performance metrics 170 for a specified EMG application may be loaded into the set of signal quality compliance algorithms 12. An analog output 17 of EMG sensor signal data 16, which includes regions of baseline noise 14 and regions of EMG signal activity 15, is digitized by an analog to digital converter 18 that outputs digitized EMG sensor signal data 19 that is processed by the set of signal quality compliance algorithms 12. Calculated results 159 of signal quality assessment from the performance comparison matrix 140 are graphically rendered 160 and presented in a visual display 240 that includes pass/fail text 200, analog and digital indicators 210, and instructional text 230. A separate Boolean value 152 of the signal quality assessment from the performance comparison matrix 140 may be provided as the digital pass/fail output 153 for process control.

The following describe in detail, the process flow for each algorithm in the set of the above mentioned signal quality compliance algorithms 12.

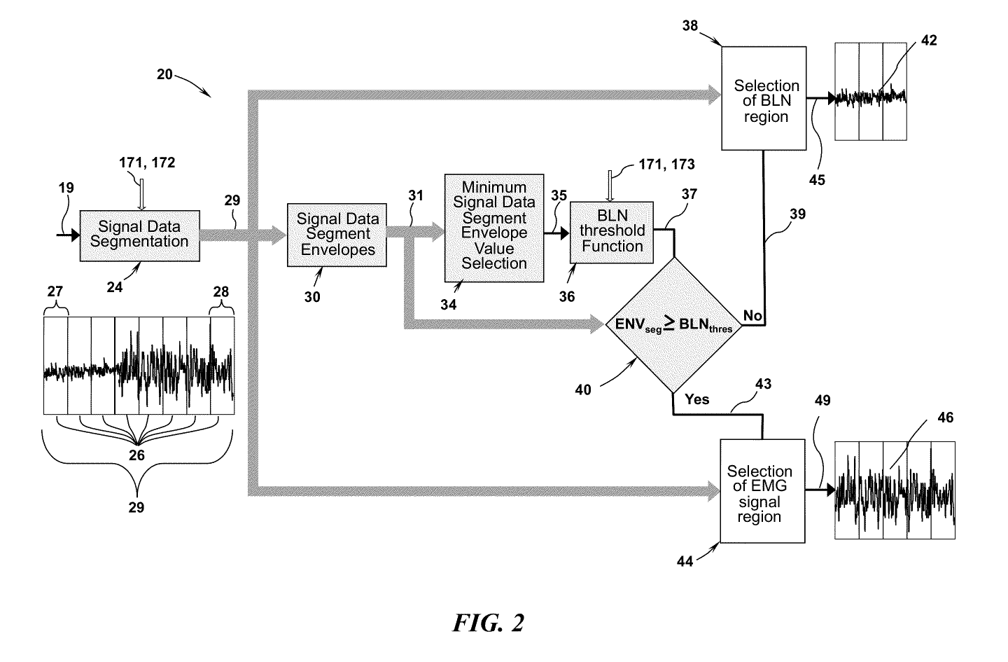

FIG. 2 details an algorithm 20 for selecting a baseline noise region 38 and an EMG signal activity region 44 of the digitized EMG sensor signal data 19. The digitized EMG sensor signal data 19 may be divided into a group of data segments 29 by a signal data segmentation function 24 of the baseline noise and EMG signal activity region selection algorithm 20. A time duration for each individual data segment 26 may be pre-defined by a segment interval metric 172 of a set of performance metrics 171. An envelope of each data segment 26 may be calculated by an EMG sensor signal data envelope function 30 of a baseline noise and EMG signal activity region selection algorithm 20, and the mean value of an envelope 31 for each data segment 26 is provided to a minimum signal data segment envelope value function 34, which determines a minimum envelope value 35 from a set of all the mean values of the envelope 31 for each of the data segments 26. A baseline noise (BLN) threshold function 36 multiplies a minimum envelope value 35 using a pre-defined multiplier value 173 metric of the set of performance metrics 171 to provide a baseline noise threshold output value 37. After the determination of the baseline noise threshold output value 37, the mean value of the envelope 31 of the first segment 27 in the series of signal data segments 29 is selected and compared to the stored BLN threshold output value 37 by a comparison function 40. The comparison function 40 determines if a mean value of the envelope of the selected data segment 31 is equal to or greater than the BLN threshold output value 37. If the result of the comparison of the envelope of selected data segment 31 is equal to or greater than the BLN threshold output value 37, then a command 43 is provided to a selection of EMG signal activity region function 44 to add the respective signal data segment 27 to an EMG signal activity region 49. If the result of the comparison of the envelop of selected data segment 31 is less than the baseline noise threshold output value 37, then a command 39 is given to a selection of baseline BLN region function 38 to add the respective signal data segment 27 to a baseline noise region 45. A segment comparison 40 like that described above is repeated for each of the segments 26 in a series of all of the signal data segments 29, until the completion of the process for a last signal data segment 28. In the case where the digitized EMG sensor signal data 19 contains only baseline noise, or only EMG signal activity, the calculated value of the minimum data segment envelope 35 may characterize all data segments above the calculated baseline noise threshold value 37 as EMG signal activity region 44 data segments, and may characterize all signal data segments below the calculated baseline noise threshold value 37 as baseline region 38 data segments. Because there may be only a small difference between the signal data segment envelope values 31 characterized as baseline noise region 38 and the signal data segment envelope values 31 characterized as EMG signal activity region 44, the SNR algorithm 87 calculation output 122 may produce a value of approximately one, which could be interpreted by the performance metrics comparison matrix 140 as a signal quality assessment failure condition at an output 152.

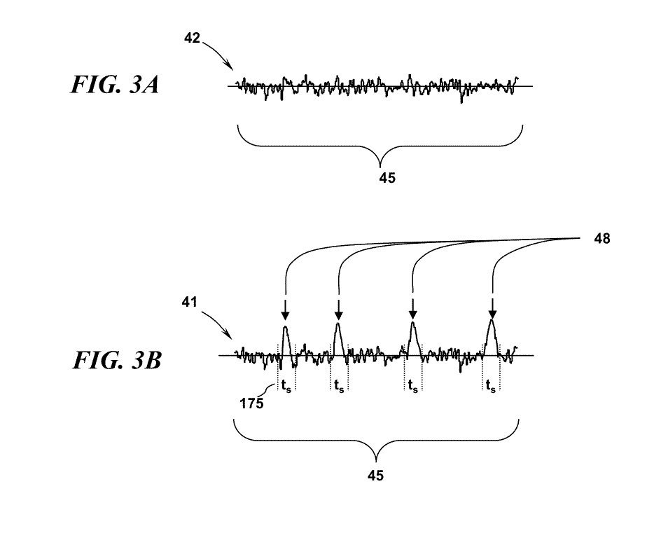

An output 45 of the selected baseline noise region of the digitized EMG sensor signal data 19 and the EMG signal activity region 49 of the EMG sensor signal data 19 may be further processed by the respective baseline noise region processing algorithms 100 and EMG signal activity region processing regions 110. FIGS. 3-7 illustrate representative examples of a time domain signal data from the output 45 of the selected baseline noise region of the EMG sensor signal data 19 and the EMG signal activity region 49 of the EMG sensor signal data 19, together with a resultant processed baseline noise region envelope 80, processed EMG signal activity region envelope 84, and respective baseline noise frequency domain and EMG signal frequency domain outputs.

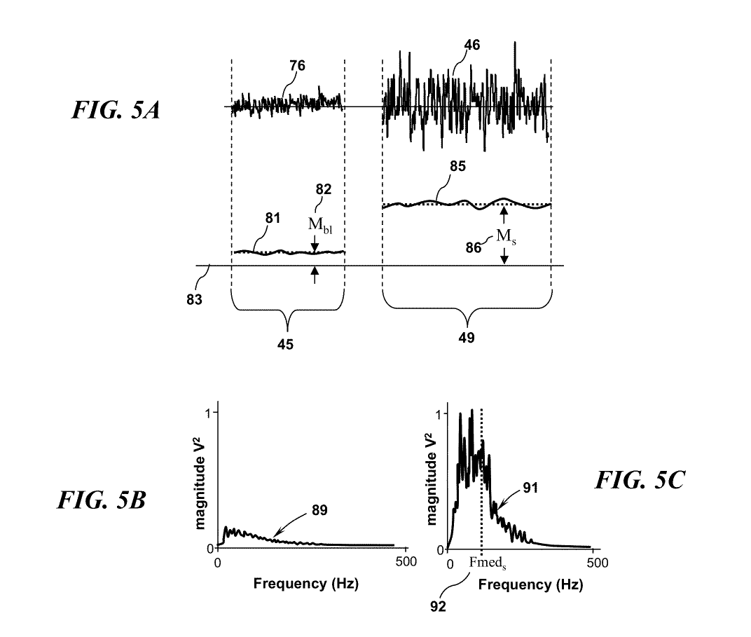

The output 45 of the selected baseline noise region of the EMG sensor signal data 19 is processed by the baseline noise region processing algorithms 100 that include a motor unit (MU) detection and removal algorithm 50, an algorithm 80 to determine the BLN signal envelope, and a BLN PSD algorithm 88. FIG. 3A shows an output wave shape 42 of the output 45 corresponding to selected baseline noise region of the EMG sensor signal data 19, uncontaminated by extraneous MU pulses or line interference. As a comparison, FIG. 3B shows an output wave shape 41 of the output 45 corresponding to the selected baseline noise region of the EMG sensor signal data 19 contaminated by extraneous MU pulses 48 present in the data. The MU pulses 48 may occur in the output 45 if muscle is not fully relaxed and can lead to an overestimation of a baseline noise envelope amplitude M_{bl} 82 shown in FIG. 5B.

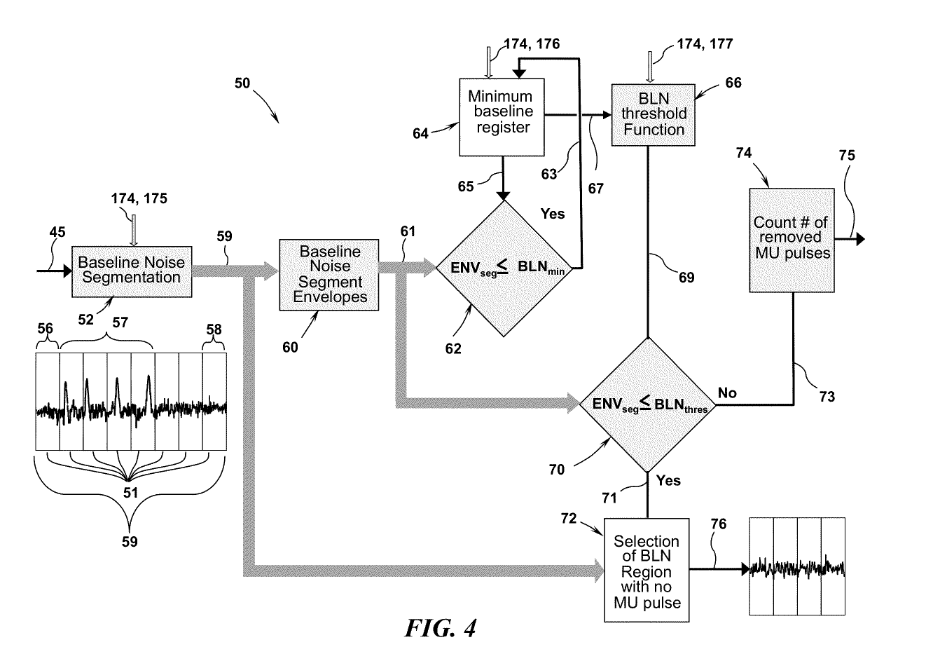

The MU detection and removal algorithm 50 determines all time segments tₛ 175 of output 45 that contain the MU pulse 48 and remove the time segments 175 from the output 45 so that proper estimation of the baseline noise envelope amplitude M_{bl} 82 and baseline PSD magnitude M_{psdlf} 97 can be calculated. The MU detection algorithm 50 determines the number 75 of the time segments tₛ 175 of the output 45 that contain the MU pulse 48 and provides the number 75 to the performance metrics comparison matrix 140. An excessive number of the MU segments 48 in the output 45 triggers a signal quality failure indication at the output 153 and the pass/fail display 200. FIG. 4 shows the major functional blocks of the MU detection algorithm 50. Note that at least some of the MUs may be based on voluntary muscle movement. The input to a baseline noise segmentation function 52 is output 45, described above. The baseline noise segmentation function segments the data according to the time segments tₛ 175 of a set of performance metrics 174. A first segment 56 in a series of signal data segments 59 is selected and the mean value of an envelope 61 of the selected segment 56 is calculated by a baseline noise segment envelope function 60. The envelope 61 of the selected segment is compared to a current value 65 stored in a minimum baseline register 64. The minimum baseline register 64 is initialized to a predefined initial minimum baseline envelope metric 176 of the set of performance metrics 174. If the envelope 61 for the selected segment is less than the current value 65 stored in the minimum baseline register 64, the minimum baseline register 64 is updated with the envelope 61 of the selected segment. A BLN threshold function 66 multiplies an updated minimum baseline register value 67 by a predefined metric for allowable baseline noise variation 177 of the set of performance metrics 174. If mean signal envelope 61 of the selected segment is less than an output of the BLN threshold function 69 at a comparison 70, then a command 71 is given to the selection of baseline region with no MU pulse function 72 to add the respective signal data segment 56 to the baseline region with MU pulses removed. If the mean signal envelope 61 of the selected segment is greater than the BLN threshold function 69, the segment is determined to contain a MU pulse and the count of MU pulses removed 75 is incremented by an MU pulse removed counter 74. The above sequence and comparison 70 is repeated for each of the segments 51 in the series of all of the baseline data segments 59, until completion of the process for a last baseline data segment 58. An output 76 of the selected baseline region of the output 45 with the MU pulse 48 removed is provided to the algorithm 80 for detecting BLN envelope, and the BLN PSD algorithm 88.

FIG. 5A shows a time domain plot of the output 45 processed to remove MU's from the output 76, and the selected EMG signal activity region with the time domain plot output 46. FIG. 5A also shows a plot of the baseline noise region envelope calculation and a plot of an EMG signal activity region envelope calculation. The algorithm used to determine the envelopes can be the root mean squared value (RMS) or the average rectified value (ARV). The mean magnitude M_{bl} 82 of a baseline envelope value 81 and a mean magnitude Mₛ 86 of an EMG signal activity envelope value 85 are determined with respect to the zero reference line 83 of the plots of the envelope calculations 80, 84. The mean magnitude M_{bl} 82 of the output 45 and the calculated envelope output 86 of the selected EMG signal activity region are used to calculate the EMG signal to baseline signal to noise ratio (SNR) using the formula:

SNR=(EMG region envelope amplitude M_{s)/(}Baseline region envelope amplitude M_{bl)}

FIG. 5B shows a plot 89 of the calculated BLN PSD of the output 45 with the selected baseline region processed to remove MU's from the output 76. FIG. 5C shows an EMG PSD plot 91 of the calculated EMG PSD of the time domain plot output 46 of the EMG signal activity region 49 shown in FIG. 5A. The algorithm used to determine the PSD of the output 45 and the EMG signal activity region 49 is based on the Welch method of determining the Discrete Fourier Transform. A median frequency Fmedₛ 92 parameter of the EMG PSD plot 91 of the EMG signal activity region 49 is also calculated. The calculated median frequency Fmedₛ 92 output of the EMG PSD signal processing algorithm is provided to the PSD compliance function 120 and the performance metrics comparison matrix 140, and is used to characterize the frequency content of the EMG signal activity region 49 of the EMG sensor signal data output 19. Although the median frequency Fmedₛ 92 parameter may be used, alternative parameters such as mean frequency can be used to characterize the spectrum of the EMG signal activity region 49.

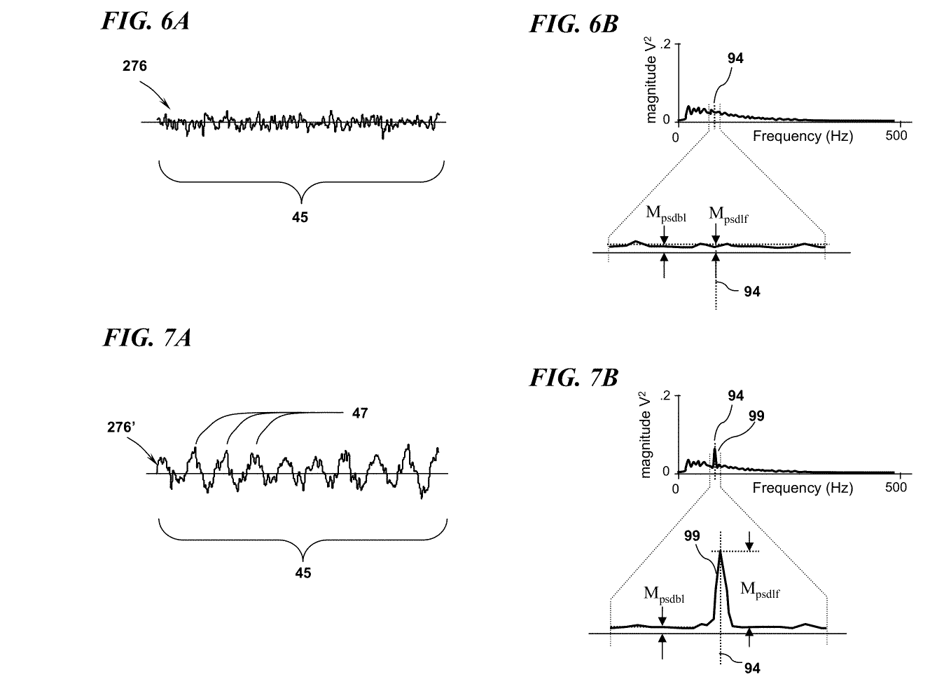

FIGS. 6A-6B and 7A-7B illustrate a process for determining a magnitude of power line contamination in the output 76 of selected baseline noise region 45. FIG. 6A shows a time domain plot 276 of the selected baseline noise region 45 that is uncontaminated by power line interference. A plot of the calculated BLN PSD of the uncontaminated selected baseline region 45 is shown in FIG. 6B, together with an expanded view of the plot centered on a frequency region of a power line frequency 94. A PSD magnitude M_{psdlf} at a frequency region of the power line frequency 94 of the plot is comparable to an average PSD magnitude M_{psdbl} in a frequency region adjacent to the frequency region of the power line frequency 94. As a comparison, FIG. 7A shows a time domain plot 276′ of the selected baseline noise region 45 that is contaminated by power line interference 47. A plot of the calculated BLN PSD of the contaminated selected baseline noise region 45 is shown in FIG. 7B together with an expanded view of the plot centered on a frequency region of the power line frequency 94. A PSD magnitude M_{psdlf} at the frequency region of the power line frequency 94 of plot shows an increase in amplitude 99 that is higher than an average PSD magnitude M_{psdbl} in the frequency region adjacent to the frequency region of the power line frequency 94. A calculated BLN PSD output of the selected baseline noise region 45 is provided to the performance metrics comparison matrix 140. An excessive PSD magnitude value M_{psdlf} at the frequency region of the power line frequency 94 in the baseline region signal data 45 triggers a signal quality failure indication at the output 153 and the pass/fail display 200.

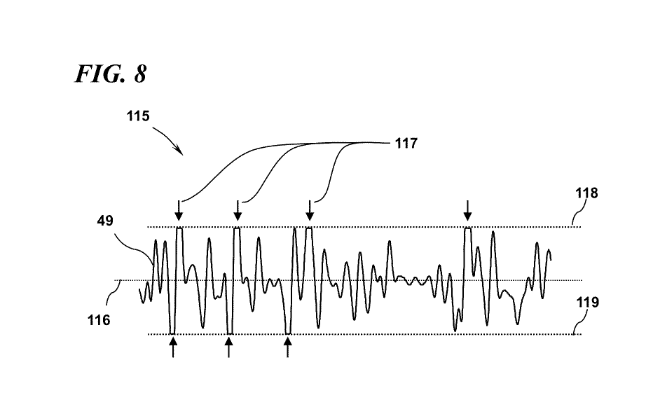

FIG. 8 shows a time domain plot of the selected EMG signal activity region 49 that is contaminated by occurrences 117 of data samples which are at a maximum positive value 118 and at a maximum negative value 119 from the digital output 19 of the A/D convertor 18. This condition is termed “clipping” and results from analog output 17 signal levels which exceed the dynamic range of the A/D converter 18. A maximum data value algorithm 115 calculates a maximum absolute data value 114 from the digital output 19 of the A/D converter 18 and provides the maximum absolute data value 114 to the performance metrics comparison matrix 140. An excessive maximum absolute data value 114 from the digital output 19 of the A/D converter 18 triggers a signal quality failure indication at the output 153 and the pass/fail display 200.

As further illustrated in FIG. 1, the set of signal quality compliance algorithms 12 include an SNR compliance function 130 and a PSD compliance function 120. The functions 120, 130 provide the ability to modify maximum acceptable values for magnitude of the selected baseline noise region BLN envelope mean magnitude M_{bl} 82, and the PSD magnitude M_{psdlf} 97 of the power line frequency 94 determined by BLN PSD algorithm 88.

In many applications, EMG sensor signals 16 with a high SNR value, can tolerate a greater degree of baseline noise region envelope mean magnitude M_{bl} 82, and the signal quality acceptance criteria for baseline noise envelope mean magnitude M_{bl} 82 can be increased. The SNR compliance function 130 uses pre-defined performance metrics values 179 from the set of pre-defined signal quality performance metrics 170 which define the relationship between baseline noise envelope value 82 and SNR value 122. The output 135 of the SNR compliance function 130 is provided to the performance metrics comparison matrix 140 to adjust the pre-defined limit for baseline noise based on SNR 122.

Similarly, EMG sensor signals 16 with a high SNR value 122 and/or a high PSD Fmed_{psd} output value 92 can tolerate a greater PSD magnitude M_{psdlf} 97 at the frequency region of the power line interference frequency 94 in the calculated BLN PSD of the selected baseline noise region signal data 45, and the signal quality acceptance criteria for power line interference value 97 can be increased. This is possible because an Fmed_{psd} value 92 greater than the PSD magnitude M_{psdlf} 97 at the frequency region of the power line interference frequency 94 allows for the implementation of a high pass or notch filtering reducing its percentage of line interference contamination to an acceptable level, especially for EMG sensor signals 16 with a high SNR 122. The PSD compliance function 120 uses pre-defined performance metrics values 178 from the pre-defined signal quality performance metrics 170 which define the relationship between the calculated PSD Fmed_{psd} value 92, the calculated SNR value 122, and the calculated PSD magnitude M_{psdlf} 97 at the frequency region of the power line interference frequency 94. The output 125 of the PSD compliance function 120 is provided to the performance metrics comparison matrix 140 to adjust the pre-defined limit for the calculated PSD magnitude M_{psdlf} 97 based on calculated SNR 122 and calculated Fmed_{psd} value 92.

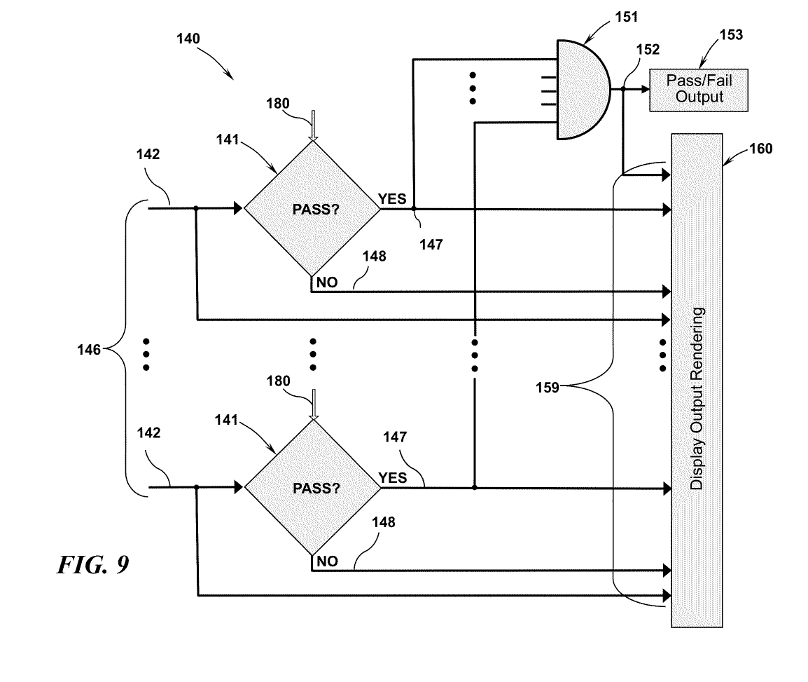

The performance metrics comparison matrix 140 shown in FIG. 1 performs a numerical comparison on each of the calculated output values of the baseline region processing algorithms 100, the EMG signal activity region processing algorithms 110, the maximum data value algorithm 115, the SNR calculation value 122, and the PSD compliance 125 and SNR compliance 130 function values with respect to their pre-defined acceptable performance metrics values 180. FIG. 9 shows the major functional blocks of the performance metrics comparison matrix 140. Each calculated output 142 in the group of calculated outputs 146 is compared with its respective predefined performance metric 180 in the performance metrics comparison matrix 140 using a separate binary pass/fail comparison function 141. For each calculated output 142, the comparison function 141 provides the binary value 1 at the YES output 147 for a PASS result and provides the binary value 1 at the NO output 148 for a FAIL result. The YES binary output 147 of each separate pass/fail comparison function 141 is provided to a multiple input Boolean AND gate 151. The binary output 152 of the multiple input Boolean AND gate 151 is a binary value 1 if each YES binary output 147 of each separate pass/fail comparison function 141 is a binary value 1 indicating the result of the signal quality assessment has passed. If any YES binary output 147 of each separate pass/fail comparison function 141 is a binary value 0, then binary value of the Boolean AND gate 151 is 0 indicating the result of the signal quality assessment has failed. The output of Boolean AND gate 152 is provided to the digital Pass/Fail output 153 and the display output rendering function 160. The results 159 of the performance metrics comparison matrix 140 consisting of the Boolean results of the numerical comparison 152 together with the calculated value of the magnitude of the baseline noise envelope 82, the calculated values of the scaled, calculated PSD magnitude 97 at the frequency region of the power line interference frequency 94, and the calculated SNR value 122 are rendered for output display 160. When all of the results 159 of all comparisons of the calculated output values meet or exceed the pre-defined acceptable performance metrics values 180, a Boolean 1 PASS output 152 and an “OK to Proceed” indication 205 on the graphical display 200 will be generated. When any of the results 159 of all comparisons of the calculated output values do not meet or exceed the pre-defined acceptable performance metrics values 180, a Boolean 0 FAIL output 152 and a FAILED indication 205 on the graphical display 200 will be generated.

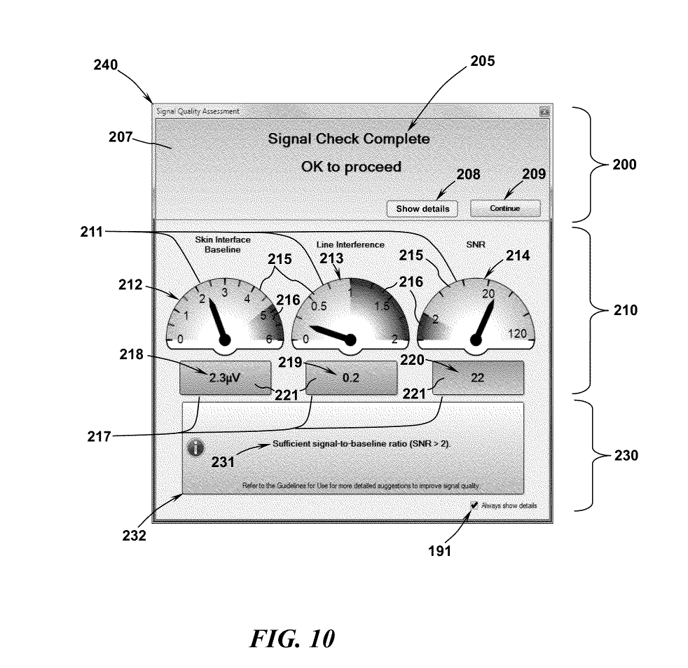

FIG. 10 shows a screen shot of the visual display 240 for the presentation of the results for a signal quality assessment which met or exceeded all pre-defined performance metrics. The display 240 is comprised a Pass/Fail display result region 200, a calculated signal quality results region 210, and an instructional text region 230. During the initial presentation of the results display 240, for a signal quality assessment which met or exceeded all pre-defined performance metrics, only the Pass/Fail display result region 200 is displayed. The calculated signal quality results region 210 and the instructional text region 230 are hidden from view, unless the “Always show details” check box 191 is selected active as is shown in this illustration. Alternatively, the calculated signal quality results region 210, and the instructional text region 230 can be made visible as a result of the operator selecting the control button 208. The Pass/Fail display result region 200 has a signal quality result text message output 205 indicating “Signal Check Complete” and that it is “OK to Proceed”. The Pass/Fail display result region 200 is filled with the color green 207 to indicate the signal quality assessment result met the pre-defined performance metrics 180 for signal quality. The “Continue” control button 209 closes the display window 240. The calculated signal quality results region 210 of the display 240 presents analog meter displays 211 consisting of an analog meter display of the baseline noise value 212, an analog meter display of the scaled power line interference value 213, and an analog meter display of the SNR value 214. Each of the analog meter displays 211 has region 215 filled with the color green to indicate the range of output values which fall within the pre-defined acceptable performance metrics values 180 and has region 216 filled with the color red to indicate the range of output values which fall outside the pre-defined acceptable performance metrics values 180. The calculated signal quality results region 210 also presents digital numerical displays 217 consisting of a digital display of the baseline noise value 218, a digital display of the scaled power line interference value 219, and a digital display of the SNR value 220. Each of the digital displays 217 has a region filled with the color green 221 to indicate the range of output values fall within the pre-defined acceptable performance metrics values 180. The instructional text region 230 of the display 240 contains a text block 232 with an instructional message 231 indicating that the SNR value is sufficient.

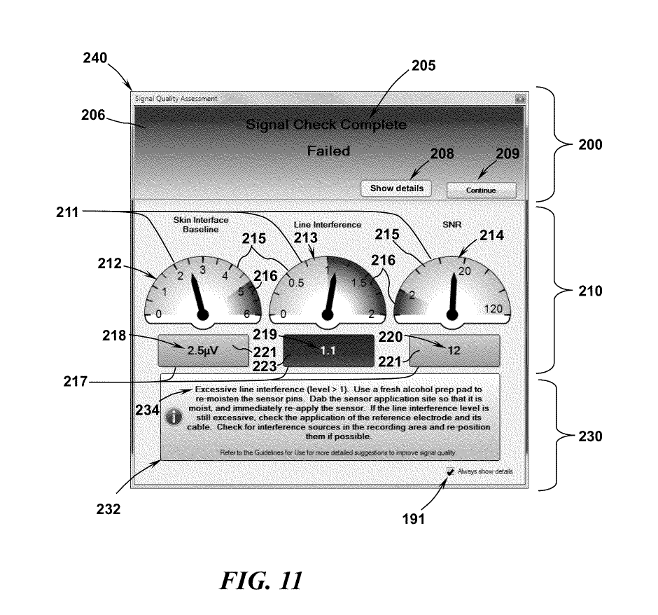

FIG. 11 shows a screen shot of the visual display 240 shown in FIG. 10 representing the results for a signal quality assessment which failed to meet one or more of the pre-defined performance metrics 180. When the results for a signal quality assessment fail to meet one or more of the pre-defined performance metrics 180, the Pass/Fail display result region 200, a calculated signal quality results region 210, and an instructional text region 230 of the display 240 are all simultaneously displayed. The Pass/Fail display result region 200 has a signal quality result text message output 205 indicating “Signal Check Complete” and that it “Failed”. The Pass/Fail display result region 200 is filled with the color red 206 to indicate a negative signal quality assessment result. Each of the digital displays 217 has region filled with the color green 221 to indicate the range of output values which fall within the pre-defined acceptable performance metrics values 180 and filled with the color red 223 to indicate the range of output values which fall outside the limits of the pre-defined acceptable performance metrics values 180. In this example, the digital display for line interference 219 has its region filled with the color red 223 indicating that the predefined metric 180 for line interference 219 has been exceeded. The instructional text region 230 of the display 240 contains a text block 232 with an appropriate instructional message 234 for addressing and correcting the failure conditions.

The process for assessing the signal quality of an EMG sensor signal data output channel described herein may be applied as a stand-alone process, or can be included as a signal processing component within a group of data acquisition and processing components used during the data acquisition function of an electromyographic application. The process described herein can also be applied as a stand-alone process, or can be included as a signal processing component within a group of signal processing components forming the data analysis function of an electromyographic application.

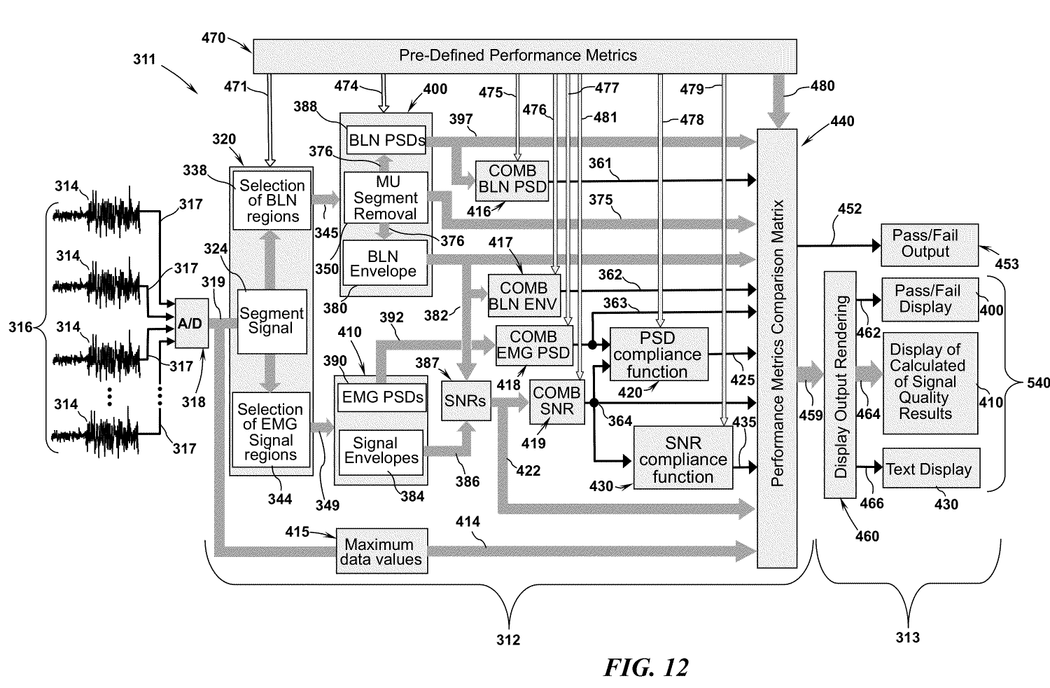

Another embodiment of a system 311 for assessing signal quality of a plurality of EMG sensor signal data output channels includes a set of signal quality compliance algorithms 312, generated visual displays of graphics and text 313, and a digital pass/fail result output 453 as illustrated in the system block diagram of FIG. 12. The system 311 provides for multi-channel input. As with the system 11, described above, the sensor data may be from a single differential EMG sensor or from a double differential EMG sensor placed on the skin of a subject. The system 311 may be implemented using hardware (e.g., off-the shelf computer processing hardware), software (e.g., a computer program written in an appropriate language to provide the functionality described herein), or some combination thereof. In an embodiment herein, the system 311 may be implemented using a computing device such as an off-the-shelf personal computer running an appropriate operating system and/or may be or may include an embedded system running in connection with a personal computer or a relatively larger computing device, such as a minicomputer. The computing device receives signal data in the form of real-time analog or digital inputs or, in some cases, may process data that has already been accumulated and stored and provided on analog or digital tape or otherwise provided as analog or digital data. The data may be provided by band pass filtering sensor data, possibly providing pre-amplification with gain and filtering. In the case of digital data, an analog to digital converter may be used. The computing device is programmed to provide the functionality described herein.

The following is a general overview of the major functional blocks of the process according to the system 311 shown in FIG. 12. The set of signal quality compliance algorithms 312 includes a multi-input baseline noise and EMG signal activity region selection algorithm 320, a group of multi-input baseline noise region signal processing algorithms 400, a group of multi-input EMG signal activity region processing algorithms 410, a multi-input maximum data value algorithm 415, a multi-input baseline region PSD combination algorithm 416, a multi-input baseline region envelope combination algorithm 417, a multi-input EMG signal activity region PSD combination algorithm 418, a multi-input SNR computation algorithm 419, a PSD compliance function 420, an SNR compliance function 430, and a performance metrics comparison matrix 440. In operation, pre-defined signal quality performance metrics 470 for a specified EMG application may be loaded into the set of signal quality compliance algorithms 312. The EMG sensor signal data channels 316 to be proceeded for signal quality can originate from multiple separate, independent sensor technologies, individual sensor array technologies with multiple signal outputs, or a combination of both technologies. The analog output 317 of each of the multiple EMG sensor signal data channels 314 is digitized by an analog to digital convertor 318 whose multi-channel digital output 319 is processed by the set of signal quality compliance algorithms 312. The calculated results 459 of the signal quality assessment from the performance comparison matrix 440 are graphically rendered 460 and presented in a visual display 540 consisting of pass/fail text 400, analog and digital indicators 410, and instructional text 430. A separate Boolean value 452 of the signal quality assessment from the performance comparison matrix 440 is provided as digital pass/fail output 453 for process control.

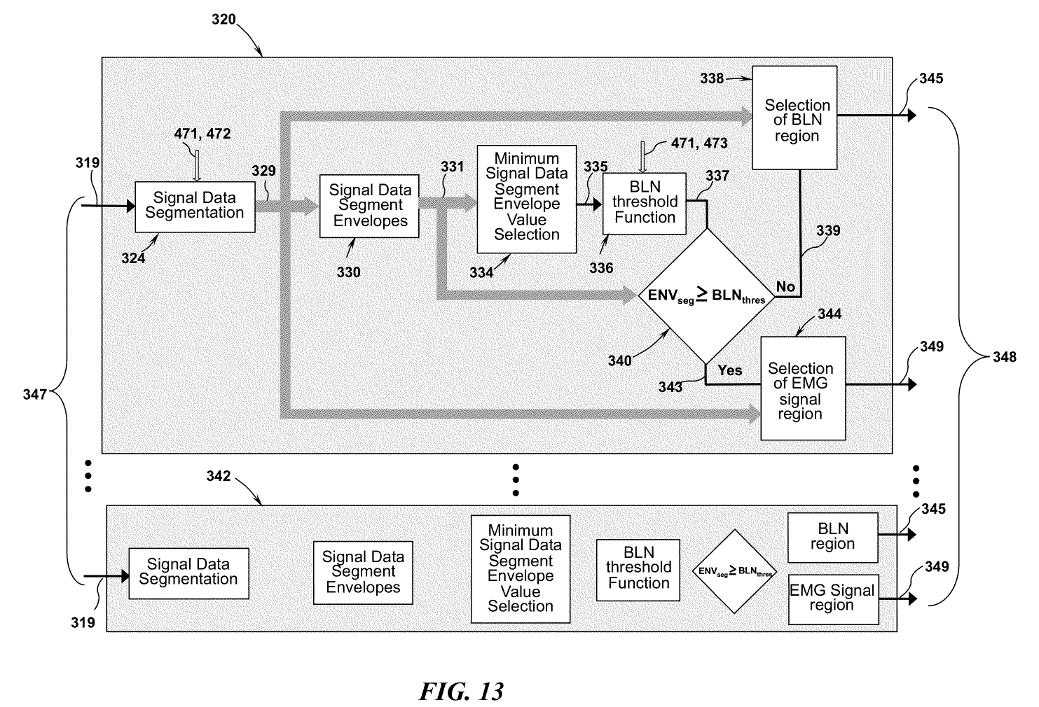

The following describes in detail a process flow for each algorithm in the set of the above mentioned signal quality compliance algorithms 312. FIG. 13 shows a shaded block diagram of the algorithm 320 used for selecting the multi-channel baseline noise region 345 and the multi-channel EMG signal activity region 349 of the digitized sensor signal data 319. For each digitized sensor signal channel 319 of the multi-channel EMG sensor signal data 347, the following sequence of steps is performed to select the regions of baseline noise 345 and to select the regions of EMG signal activity 349. The digitized signal data 319 is divided into data segments 329 by the multi-channel signal data segmentation function 324 of the multi-channel baseline noise and EMG signal activity region selection algorithm 320. The time duration for each data segment is pre-defined by the segment interval metric 472 of the set of performance metrics 471. The envelope of each data segment 331 is calculated by the signal data envelope function 330 of the baseline noise and EMG signal activity region selection algorithm 320, and the mean value of the envelope 331 for each data segment 329 is provided to the minimum signal data segment envelope value function 334 which determines the minimum envelope value 335 from the set of all the mean values of the envelope segments 331. The baseline BLN threshold function 336 multiplies the minimum envelope value 335 by a pre-defined multiplier value 473 metric of the set of performance metrics 471 to provide a baseline noise threshold output value 337. After the determination baseline noise threshold output value 337, the mean value of the envelope 331 of the first segment in the series of signal data segments 329 is selected and compared to the stored baseline noise threshold output value 337 by the comparison function 340. The comparison function 340 determines if the mean value of the envelope of the selected data segment 331 is equal to or greater than the baseline noise threshold output value 337. If the result of the comparison of the envelop of selected data segment 331 is equal to or greater than the baseline noise threshold output value 337, then a command 343 is given to the selection of EMG signal activity region function 344 to add the respective signal data segment 329 to the EMG signal activity region 349. If the result of the comparison of the envelop of selected data segment 331 is less than the baseline noise threshold output value 337, then a command 339 is given to the selection of baseline region function 338 to add the respective signal data segment 329 to the baseline region 345. For each the channels 347, the above comparison 340 is repeated for each segment in the series of all of the EMG sensor signal data segments 329, until the completion of the process for the last signal data segment, and the process 320 repeated until completion of the last channel 342.

As shown in FIG. 12, the time domain signal data from each channel of the multi-channel outputs 345 of the selected baseline region of the EMG sensor signal data 338 and the multi-channel outputs 349 of the selected EMG signal activity region of the EMG sensor signal data 344 are further processed by their respective group of multi-channel baseline region processing algorithms 400 and group of multi-channel EMG signal activity region processing algorithms 410. The group of multi-channel baseline region processing algorithms 400 is comprised of a multi-channel motor unit (MU) detection and removal algorithm 350, a multi-channel algorithm to determine the baseline noise signal BLN envelope 380, and a multi-channel BLN PSD algorithm 388. The group of multi-channel EMG signal activity region processing algorithms 410 is comprised of a multi-channel EMG PSD algorithm 390, and a multi-channel algorithm to determine the EMG signal activity envelope 384. The function of the multi-channel MU detection and removal algorithm 350 of the baseline region processing algorithms 400 is to determine all the presence of MU pulses in each the selected baseline regions of the respective signal data channels 345 remove these MU pulses from the baseline regions of the respective signal data channels 345 so that proper estimation of baseline noise envelope amplitude 382 and BLN PSD amplitude 397 for each respective channel can be calculated. The MU detection and removal algorithm 350 determines the number of MU pulses 375 in the baseline regions of the respective signal data channels 345 and provides this number for each respective output to the performance metrics comparison matrix 440. Note that at least some of the MUs may be a result of voluntary muscle movement. An excessive number of MU pulses 375 in the baseline region signal data 345 compared to the pre-defined metric for acceptable number of MU segments 480 will trigger a signal quality failure indication on the pass/fail output 453 and on the visual displays 540. The multi-channel output 376 of the selected baseline regions of the respective signal data channels with the respective MU pulses removed is provided to the multi-channel baseline noise signal BLN envelope algorithm 380, and the multi-channel BLN PSD algorithm 388. The algorithm used to determine the multi-channel envelopes 380 of the selected baseline noise regions with the respective MU pulses removed 376, and multi-channel envelopes 384 of selected EMG signal regions 349 can be the root-mean-squared value (RMS) or the average rectified value (ARV) calculation. The magnitude of each of the calculated multi-channel baseline noise region envelope values 382 and the magnitude of each of the calculated multi-channel the EMG signal activity envelope values 386 are used to calculate the multi-channel EMG signal to baseline noise SNR 387 of each of the respective channels. The algorithm used to determine the multi-channel PSD of the selected baseline 388 and multi-channel PSD of the selected EMG signal activity 390 regions is based on the Welch method of determining the Discrete Fourier Transform. A multi-channel BLN PSD algorithm 388 is used to calculate the magnitude of power line contamination at the frequency regions of the power line frequency component in the output of the power spectrum 388 of the selected baseline region 376 of each of the respective channels. The value of the calculated multi-channel output 397 of each of the respective channels of the multi-channel PSD algorithm 388 is provided to the performance metrics comparison matrix 440 and the BLN PSD combination algorithm 416. A multi-channel EMG PSD algorithm 390 is used to calculate the median frequency 392 output of the power spectrum of the selected EMG signal activity region 349 of each EMG sensor signal data channels 316. Although the median frequency parameter may be used, alternative parameters such as mean frequency can be used to characterize the EMG signal activity region 349 spectrum. The median frequency output 392 of each channel is provided to the EMG PSD combination algorithm 418. For each of the EMG sensor data channels 316, the maximum data value algorithm 415 calculates the maximum absolute data value 414 from the digital output 319 of the A/D converter 318 and provides this output 414 to the performance metrics comparison matrix 440. An excessive maximum absolute data value 414 from the digital output 319 of the A/D converter 318 will trigger a signal quality failure indication at the digital output 453 and the pass/fail displays 540.

As further illustrated in FIG. 12, the set of signal quality compliance algorithms 312 in the system 311 includes a BLN PSD combination algorithm 416, a BLN envelope combination algorithm 417, an EMG signal activity PSD combination algorithm 418, an SNR combination algorithm 419, a PSD compliance function 420, and an SNR compliance function 430.

In addition to the ability of the performance metrics comparison matrix 440 to compare the processed values obtained from each individual channel output, the signal quality assessment process 312 can combine the respective outputs of the multi-channel baseline signal BLN PSD algorithm 388, the multi-channel baseline signal envelope algorithm 380, the multi-channel EMG signal activity PSD algorithm 390, and the multi-channel SNR algorithm 387 into a single representative output value for each algorithm. This feature of the system described herein is useful for characterizing the overall signal quality performance from array sensors whose multiple signal channels share a common detection region and share common signal amplitude and frequency attributes.

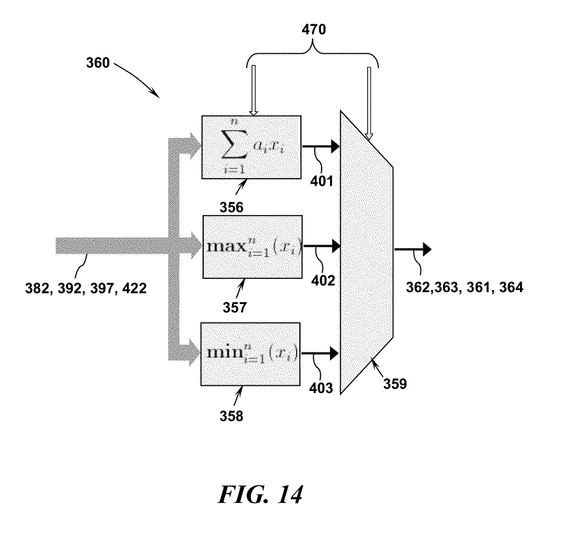

FIG. 14 shows a block diagram of a generic implementation of a multi-channel combination algorithm 360 used to illustrate the approach and implementation of the common functional elements utilized by the baseline BLN PSD combination algorithm 416, the baseline envelope combination algorithm 417, the EMG signal activity PSD combination algorithm 418, and the SNR combination algorithm 419. The inputs to the generic algorithm 360 represent the respective calculated outputs of each of the channels from the multi-channel baseline PSD algorithm 397, the multi-channel baseline envelope algorithm 382, the multi-channel EMG activity PSD algorithm 392, and the multi-channel SNR algorithm 422. The common shared functional elements of the generic combination algorithm 360 consist of a weighted average function 356, a maximum value function 357, a minimum value function 358, and a modality function 359 that determines the selected function output 362, 363, 361, and 364. The features of the generic combination algorithm 360 as they relate to each implementation are described in the following sections.

The baseline BLN PSD combination algorithm 416 calculates an output value that can be the weighted average of the magnitude of power line contamination at the frequency regions of the power line frequency component in the output signal 397 of each of the channels, the minimum value of the magnitude of power line contamination at the frequency regions of the power line frequency component in the output signal 397 of each of the channels, or the maximum value of the magnitude of power line contamination at the frequency regions of the power line frequency component in the output signal 397 of each of the channels. The type of calculation modality selected for combining magnitude of power line contamination at the frequency regions of the power line frequency component in the output signal 397 of each of the channels, is specified by the set of pre-defined performance metrics 475 and is based on type of sensor data. For individual EMG channels obtained from multiple sensors, the minimum value calculating modality 358 would typically be selected to determine the combined magnitude of power line contamination at the frequency regions of the power line frequency component 397 in the output frequency spectrum 397. For an array sensor with multiple output channels, the weighted average value calculating modality, or the minimum value calculating modality would typically be selected. The output 361 of the PSD combination algorithm is provided to the performance metrics comparison matrix algorithm 440. The baseline noise BLN envelope combination algorithm 417 calculates an output value that can be the weighted average of the magnitude of baseline noise envelope 382 of each of the channels, the minimum value of the magnitude of baseline noise envelope 382 of each of the channels, or the maximum value of the magnitude of the magnitude of baseline noise envelope 382 of each of the channels. The type of calculation modality selected for combining the magnitude of baseline noise envelope 382 of each of the channels, is specified by the set of pre-defined performance metrics 476 and is based on type of sensor data. For individual EMG channels obtained from multiple sensors, the minimum value calculating modality 358 would typically be selected to determine the combined magnitude of the baseline envelope 382 of each of the channels. For an array sensor with multiple output channels, the weighted average value calculating modality 356, or the minimum value calculating modality 358 would typically be selected. The output 362 of the baseline envelope combination algorithm 417 is provided to the performance metrics comparison matrix algorithm 440. The EMG signal activity PSD combination algorithm 418 calculates an output value that can be the weighted average of the median frequency output 392 of each of the channels, the minimum value of the median frequency output 392 of each of the channels, or the maximum value of the median frequency output 392 of each of the channels. The type of calculation modality selected for combining the median frequency output 392 of each channel of the EMG activity PSD algorithm 390 is specified by the set of pre-defined performance metrics 477 and is based on type of sensor data. For individual EMG channels obtained from multiple sensors, the minimum value calculating modality 358 would typically be selected to determine the magnitude of the combined median frequency output value 363. For an array sensor with multiple output channels the weighted average value calculating modality 356, or the minimum value calculating modality 358 would typically be selected. The output 363 of the PSD combination algorithm is provided is provided to the PSD compliance function 420 and to the performance metrics comparison matrix algorithm 440. The SNR combination algorithm 419 calculates an output value that can be the weighted average of the SNR value 356 of each of the channels, the minimum value of the SNR value 358 of each of the channels, or the maximum value of the of the SNR value 357 of each of the channels. The type of calculation modality selected for combining the SNR value 422 of each of the channels, is specified by the set of pre-defined performance metrics 481 and is based on type of sensor data. For individual EMG channels obtained from multiple sensors, the minimum value calculating modality would typically be selected to determine the combined SNR value 364. For an array sensor with multiple output channels, the weighted average value calculating modality 356, or the minimum value calculating modality 358 would typically be selected. The output 364 of the SNR combination algorithm is provided to the PSD compliance function 420, the SNR compliance function 430 and to the performance metrics comparison matrix algorithm 440.

The set of signal quality compliance algorithms 312 in the system 311 includes a PSD compliance function 420, and an SNR compliance function 430. The functions 420, 430 provide an ability to modify maximum acceptable values for magnitude of the selected baseline noise envelope 362, 382, and the magnitude of the power line frequency 361, 397 of the selected baseline PSD output 388.

In many applications, EMG sensor signals 316 with a high SNR value 422, can tolerate a greater degree of baseline noise envelope value 382, and the signal quality acceptance criteria for baseline envelope value 382 can be increased. The SNR compliance function 430 uses pre-defined performance metrics values 479 from the set of pre-defined performance metrics values 470 which define the relationship between combined baseline envelope value 362 and the combined SNR value 364. The output 435 of the SNR compliance function 430 is provided to the performance metrics comparison matrix 440 to adjust the pre-defined limit for the combined baseline envelope 361 based on combined SNR output 364.

Similarly, EMG signals 316 with a high SNR value 422 and/or a EMG PSD Fined value 392 can tolerate a greater BLN PSD magnitude 397 at the frequency region of the power line interference frequency in the calculated PSD output 397 of the selected baseline noise region signal data 376, and the signal quality acceptance criteria for power line interference value can be increased. This is possible because an EMG PSD Fined value 392 greater than the BLN PSD magnitude 397 at the frequency region of the power line interference frequency allows for the implementation of a high pass or notch filtering, reducing its percentage of line interference contamination to an acceptable level, especially for EMG signals 316 with a high SNR 422. The PSD compliance function 420 uses pre-defined performance metrics values 478 from the set of pre-defined performance metrics values 470 which define the relationship between the combined EMG PSD Fined value 363, the combined SNR value 364, and the combined BLN PSD magnitude 361 at the frequency region of the power line interference frequency. The output 425 of the PSD compliance function 420 is provided to the performance metrics comparison matrix 440 to adjust the pre-defined limit for the calculated combined BLN PSD magnitude 361 based on calculated combined SNR 364 and calculated combined EMG PSD Fined value 363.

For each channel 314 of the multi-channel EMG sensor signal data channels 316, the performance metrics comparison matrix 440 performs a numerical comparison on each of the calculated output values of the baseline region processing algorithms 400, the EMG signal activity region processing algorithms 410, the SNR calculation value 422, the PSD compliance 425, and SNR compliance 435 function values with respect to their pre-defined acceptable performance metrics values 480. The output 459 the performance metrics comparison matrix 440 consisting of the Boolean results of the numerical comparison 452 together with the calculated value of the magnitude of the baseline noise envelope 382, the calculated values of the scaled, calculated PSD magnitude 397 at the frequency region of the power line interference frequency, and the calculated SNR value 422 are rendered for output display 460.

When all comparisons of the calculated output values of the baseline region processing algorithms 400, the EMG signal region processing algorithms 410, the SNR calculation value 422, and the PSD compliance 425 and SNR compliance 430 function values with respect to their pre-defined acceptable performance metrics values 480 meet or exceed the pre-defined acceptable performance metrics values 480, a Boolean 1 (Pass) output 452, and a “OK to Proceed” indication 505 on the graphical displays 540 will be generated. When any of the comparisons of the calculated output values of the baseline region processing algorithms 400, the EMG signal region processing algorithms 410, the SNR calculation value 422, and the PSD compliance 425 and SNR compliance 430 function values with respect to their pre-defined acceptable performance metrics values 480 does not meet or exceed the pre-defined acceptable performance metrics values 480, a Boolean 0 (Fail) output 452 and a “Failed” indication 505 on the graphical display 540 will be generated.

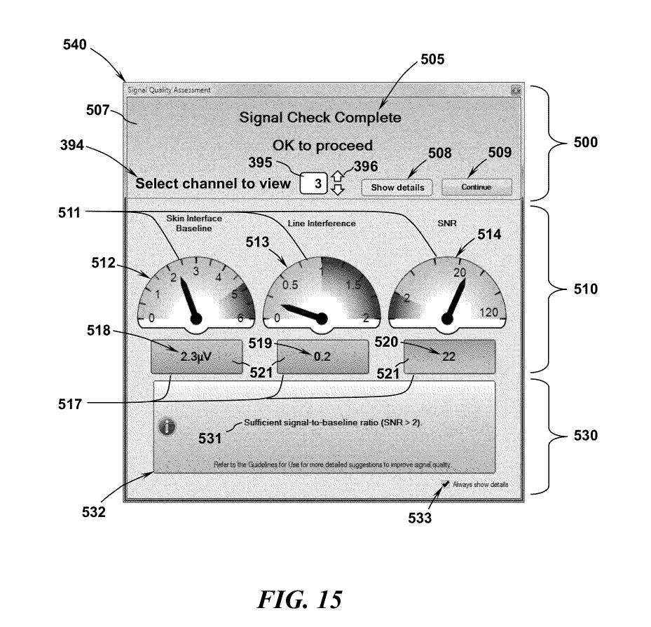

FIG. 15 shows a screen shot of the visual display 540 for the presentation of the results for a multi-channel EMG sensor signal quality assessment which met or exceeded all pre-defined performance metrics. The display 540 is comprised a Pass/Fail display result region 500, a calculated signal quality results region 510, and an instructional text region 530. During the initial presentation of the display 540, only the Pass/Fail display result region 500 is displayed with the calculated signal quality results region 510, and the instructional text region 530 hidden from view, unless the “Always show details” check box 533 is selected active as is shown in this illustration. Alternatively, the calculated signal quality results region 510, and the instructional text region 539 can be made visible as a result of the operator pressing the show details control button 508. The Pass/Fail display result region 500 has a signal quality result text message output 505 indicating “Signal Check Complete” and that it is “OK to Proceed”. The Pass/Fail display result region 500 is filled with the color green 507 to indicate the signal quality assessment result met the pre-defined performance metrics 480 for signal quality. The desired channel 395 of signal quality results for each channel 314 of the multi-channel EMG sensor channels 316 can be selected for viewing 394 in using the channel up/down control 396. The results for the selected channel 395 are viewed in the calculated signal quality results region 510 of the display. The calculated signal quality results region 510 of the display 540 presents analog meter displays 511 consisting of an analog meter display of the baseline noise value 512, an analog meter display of the power line interference value 513, and an analog meter display of the SNR value 514. The calculated signal quality results region 510 also presents digital numerical displays 517 consisting of a digital display of the baseline noise value 518, a digital display of the power line interference value 519, and a digital display of the SNR value 520. Each of the digital displays 517 has region filled with the color green 521 to indicate the range of output values fall within the pre-defined acceptable performance metrics values 480. The instructional text region 530 of the display 540 contains a text block 532 with instructional message 531.

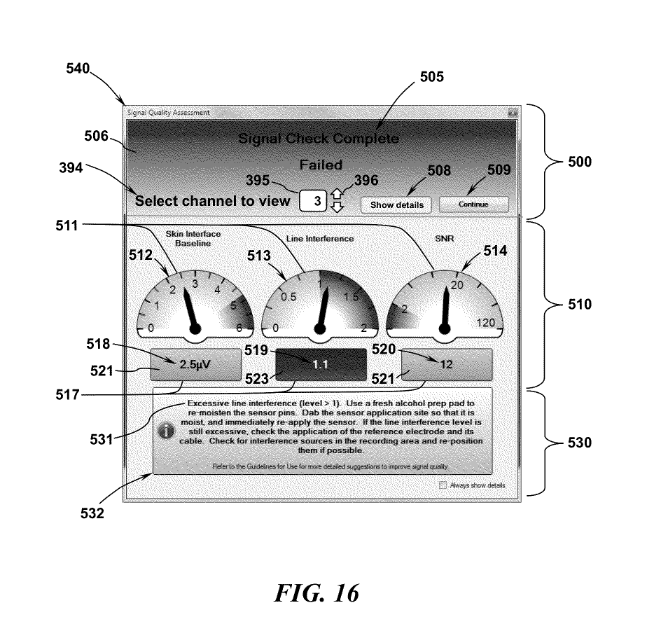

FIG. 16 shows a screen shot of the visual display 540 shown in FIG. 15 representing the results for a multi-channel EMG sensor signal quality assessment which failed to meet one or more of the pre-defined performance metrics 480. When the results for a signal quality assessment fail to meet one or more of the pre-defined performance metrics 480, the Pass/Fail display result region 500, the calculated signal quality results region 510, and the instructional text region 530 of the display 540 are all simultaneously displayed. The Pass/Fail display result region 500 has a signal quality result text message output 505 indicating “Signal Check Complete” and that it “Failed”. The Pass/Fail display result region 500 is filled with the color red 506 to indicate a negative signal quality assessment result. The results for the first channel that failed to meet one or more of the pre-defined performance metrics 480 are viewed in the calculated signal quality results region 510 of the display. Signal quality results for each additional channel 314 of the multi-channel EMG sensor channels 316 can be selected for viewing 394 in using the channel up/down control 396. The results for the selected channel 395 are viewed in the calculated signal quality results region 510 of the display. Each of the digital displays 517 has region filled with the color green 521 to indicate the range of output values which fall within the pre-defined acceptable performance metrics values 480 and filled with the color red 523 to indicate the range of output values which fall outside the limits of the pre-defined acceptable performance metrics values 480. In this example, the digital display for line interference 519 has its region filled with the color red 523 indicating that the predefined metric for line interference has been exceeded. The instructional text region 530 of the display 540 contains a text block 532 with an appropriate instructional message 531 for addressing and correcting the failure conditions.

According to the system described herein, the process for assessing the signal quality of a plurality of EMG sensor signal data output channels in the system 311 can be applied as a stand-alone process, or can be included as a signal processing component within a group of data acquisition and processing components used during the data acquisition function of an electromyographic application. The mechanism illustrated by the system 311 may also be applied as a stand-alone process, or can be included as a signal processing component within a group of signal processing components forming the data analysis function of an electromyographic application.

Various embodiments discussed herein may be combined with each other in appropriate combinations in connection with the system described herein. Additionally, in some instances, the order of steps of described flow processing may be modified, where appropriate. Subsequently, elements and areas of screen described in screen layouts may vary from the illustrations presented herein. Further, various aspects of the system described herein may be implemented using software, hardware, a combination of software and hardware and/or other computer-implemented modules or devices having the described features and performing the described functions.

Software implementations of the system described herein may include executable code that is stored in a computer readable medium and executed by one or more processors. The computer readable medium may be non-transitory and include a computer hard drive, ROM, RAM, flash memory, portable computer storage media such as a CD-ROM, a DVD-ROM, a flash drive, an SD card and/or other drive with, for example, a universal serial bus (USB) interface, and/or any other appropriate tangible or non-transitory computer readable medium or computer memory on which executable code may be stored and executed by a processor. The system described herein may be used in connection with any appropriate operating system.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method for evaluating at least one of a plurality of EMG sensor signal data outputs, comprising:
determining regions of baseline noise, line interference, and summated motor unit action potential components for each of the plurality of the signal data outputs;
arithmetically combining corresponding time and frequency domain parameters of each region into parameters to provide a set of calculated electromyographic signal performance metrics that include a baseline noise value, a magnitude and power spectra of a summated motor unit action potential components, a line interference spectra value, an EMG signal to baseline noise ratio, and a maximum data value;
visually displaying the calculated electromyographic signal performance metrics;
comparing the calculated electromyographic signal performance metrics with a set of pre-defined electromyographic signal performance metrics values;
providing an output indicating an acceptable signal quality result in response to all of the calculated performance metrics meeting the pre-defined electromyographic signal performance metrics; and
providing an output indicating a non-acceptable signal quality result in response to at least one of the calculated performance metrics not meeting the pre-defined performance metrics.

2. A method according to claim 1, wherein there is one EMG sensor signal data output.

3. A method according to claim 1, wherein there is more than one EMG sensor signal data output.

4. A method according to claim 1, wherein the set of pre-defined electromyographic signal performance metrics are determined according to performance requirements of a selected EMG application, and include an allowable number of segments from a baseline region containing motor unit action potentials, an allowable value for the EMG signal to baseline noise ratio, an allowable maximum value for a signal data output, values of coefficients of variables used in mathematical functions that arithmetically combine a plurality of individual parameter values into single respective parameter values, values of coefficients of variables used in a mathematical function that calculates an allowable baseline noise value based on the EMG signal to baseline noise ratio, and values of coefficients of variables used in a mathematical function that calculates an allowable value for spectral components of the line interference based on a calculated value of the EMG signal to baseline noise ratio and a calculated value of the summated motor unit action potential components power spectra.

5. A method according to claim 1, wherein determination of the summated motor unit action potential component includes an algorithm designed to identify data segments containing motor unit action potentials and calculate a summated motor unit action potential components region signal envelope and wherein the magnitude of the summated motor unit action potential components region signal envelope is included in the calculated performance metrics.

6. A method according to claim 1, wherein determination of the line interference component includes an algorithm designed to calculate a power spectral density function of the baseline noise and to identify magnitudes of 50 Hz, 60 Hz, and associated harmonic components of the EMG sensor signal data and wherein the magnitudes of the components of line interference are included in the calculated performance metrics.

7. A method according to claim 1, wherein determining regions of baseline noise include an algorithm designed to divide a signal envelope of the summated motor unit action potential component by a baseline noise region signal envelope and provide a result thereof as the signal to baseline noise ratio and wherein a magnitude of the signal to baseline noise ratio is included in the calculated performance metrics.

8. A method according to claim 1, wherein determination of the maximum data value includes an algorithm designed to calculate a maximum absolute value of the data and wherein the maximum absolute value is included in the calculated performance metrics.

9. A method according to claim 1, wherein the calculated performance metrics include a mean value of a magnitude of a baseline noise region signal envelope, a mean value of a line interference spectra magnitude, and a mean value of the signal to baseline noise ratio, wherein each of the mean values is calculated from a plurality of individual parameter values.

10. A method according to claim 1, wherein an allowable value for a baseline noise signal envelope is calculated as a function of a pre-defined value of allowable baseline noise and the calculated signal to noise ratio.

11. A method according to claim 1, wherein an allowable value for the spectral components of the line interference is calculated as a function of a pre-defined value of allowable spectral components of the line interference and the calculated signal to noise ratio.

12. A method according to claim 1, wherein a compliance state of a recorded signal quality result output is provided as an accessible digital control output available for integration with other hardware and software processes.

13. A method according to claim 1, wherein a visual graphic display presentation of results output are expanded by user activated control, to include presentation of calculated parameter values of the baseline noise, line interference, and signal to baseline noise ratio, using a combination of digital and analog indicators, each marked with a value of the pre-defined performance metric for respective output parameters thereof, wherein the visual graphic display presentation includes presentation of additional descriptive text blocks associated with each respective calculated parameter value output.

14. A method according to claim 13, wherein the visual graphic display presentation of results output is automatically expanded to include presentation of calculated parameter values of the baseline noise magnitude, line interference magnitude and signal to baseline noise magnitude using a combination of digital and analog indicators, each marked with the value of the pre-defined performance metric for respective output parameters thereof.

15. A method according to claim 14, wherein the visual graphic display includes presentation of additional descriptive text blocks associated with each respective calculated parameter value output having text content based on context determined by a state of compliance with a set of pre-defined performance metrics values.

16. A method according to claim 15, wherein the descriptive text blocks provide instructions for addressing and correcting conditions of non-compliance of each calculated parameter value output with respect to the set of pre-defined performance metrics values.

17. Computer software, provided in a non-transitory computer-readable medium, that evaluates at least one of a plurality of EMG sensor signal data outputs, the software comprising:
executable code that determines regions of baseline noise, line interference, and summated motor unit action potential components for each of the plurality of the signal data outputs;
executable code that arithmetically combines corresponding time and frequency domain parameters of each region into parameters to provide a set of calculated electromyographic signal performance metrics that includes a baseline noise value, a magnitude and power spectra of a summated motor unit action potential components, a line interference spectra value, an EMG signal to baseline noise ratio, and a maximum data value;
executable code that visually displays the calculated electromyographic signal performance metrics;
executable code that compares the calculated electromyographic signal performance metrics with a set of pre-defined electromyographic signal performance metrics values;
executable code that provides an output indicating an acceptable signal quality result in response to all of the calculated performance metrics meeting the pre-defined electromyographic signal performance metrics; and
executable code that provides an output indicating a non-acceptable signal quality result in response to at least one of the calculated performance metrics not meeting the pre-defined performance metrics.

18. Computer software according to claim 17, wherein the set of pre-defined electromyographic signal performance metrics are determined according to performance requirements of a selected EMG application, and include an allowable number of segments from a baseline region containing motor unit action potentials, an allowable value for the EMG signal to baseline noise ratio, an allowable maximum value for a signal data output, values of coefficients of variables used in mathematical functions that arithmetically combine a plurality of individual parameter values into single respective parameter values, values of coefficients of variables used in a mathematical function that calculates an allowable baseline noise value based on the EMG signal to baseline noise ratio, and values of coefficients of variables used in a mathematical function that calculates an allowable value for spectral components of the line interference based on a calculated value of the EMG signal to baseline noise ratio and a calculated value of the summated motor unit action potential components power spectra.

19. Computer software according to claim 17, wherein a compliance state of a recorded signal quality result output is provided as an accessible digital control output available for integration with other hardware and software processes.

20. Computer software according to claim 17, further comprising:
executable code that provides a visual graphic display presentation of results output that are expanded by user activated control to include presentation of calculated parameter values of the baseline noise, line interference, and signal to baseline noise ratio, using a combination of digital and analog indicators, each marked with a value of the pre-defined performance metric for respective output parameters thereof, wherein the visual graphic display presentation includes presentation of additional descriptive text blocks associated with each respective calculated parameter value output.
